(19) United States
(12) Patent Application Publication
(10) Pub. No.: US 2024/0376430 A1
(43) Pub. Date: Nov. 14, 2024

(54) SYSTEMS AND METHOD FOR PRODUCING T-CELLS

(71) Applicant: Terumo BCT, Inc., Lakewood, CO (US)

(72) Inventors: Dalip SETHI, Thornton, CO (US); Mindy M. MILLER, Lakewood, CO (US); Nathan SCHWAB, Lakewood, CO (US); Nathan D. FRANK, Arvada, CO (US); Mark E. JONES, Littleton, CO (US)

(21) Appl. No.: 18/657,020

(22) Filed: May 7, 2024

Related U.S. Application Data
(60) Provisional application No. 63/465,909, filed on May 12, 2023.

(51) Int. Cl. C12N 5/0783; C12N 5/00

(52) U.S. Cl. CPC C12N 5/0636; C12N 5/0081

(57) ABSTRACT

A method for producing T-cells using a cell expansion system includes expanding T-cells using a small bioreactor of the cell expansion system, the small bioreactor having a surface area of about 2,000 cm² and an intracapillary volume of about 58 milliliters. The method further includes causing T-cells to flow into a small bioreactor of the cell expansion system. A rate at which the T-cells flow into the small bioreactor may be greater than or equal to about 0.007 μL/min/fiber to less than or equal to about 0.0281 μL/min/fiber. The cell expansion system may further include a tubing set that is in fluid communication with the small bioreactor and a volume ratio of the tubing set to the bioreactor may be about 2.96.

### CROSS-REFERENCE TO RELATED APPLICATIONS

This application claims the benefit of U.S. Provisional Application No. 63/465,909 filed on May 12, 2023. The entire disclosure of the above application is incorporated herein by reference.

### FIELD

The present disclosure relates to systems and methods for producing t-cells.

### BACKGROUND

This section provides background information related to the present disclosure which is not necessarily prior art.

T-cells are white blood cells that play a central role in adaptive immune responses and are each interesting candidates for various cell therapies and treatments of various diseases, for example, where the therapies and treatments focus on harvesting and supporting the power of the existing immune system. For oncologic immunotherapies, central memory T-cells (Tcm) and stem cell memory T-cells (Tscm) have been shown to provide optimal therapeutic benefits at least in part because of the cells ability to serve as long-lived reservoirs or progenitor cells that can be homeostatically maintained and also differentiated into potent effector cells that quickly eliminate cancerous cells. Despite their promise, widespread clinical success of T-cell therapies and/or treatments have been limited because of challenges in easily and efficiently manufacturing large doses of the T-cells. Current methods for T-cell expansion are often flask-based, which can be time consuming and expensive and generally have relatively low success rates. It would be desirable to develop systems and methods for T-cell expansion having improve success rates and are also less time-consuming and expensive.

### SUMMARY

This section provides a general summary of the disclosure and is not a comprehensive disclosure of its full scope or all of its features.

In various aspects, the present disclosure provides a method for producing T-cells using a cell expansion system.

In at least one example embodiment, the method may include expanding T-cells using a small bioreactor of the cell expansion system, where the small bioreactor has a surface area of about 2,000 cm².

In at least one example embodiment, the small bioreactor has an intracapillary volume of about 58 milliliters.

In at least one example embodiment, the cell expansion system may further include a tubing set that is in fluid communication with the small bioreactor and a volume ratio of the tubing set to the bioreactor may be about 2.96.

In at least one example embodiment, the method may further include placing the tubing set into fluid communication with the small bioreactor.

In at least one example embodiment, the method may further include causing T-cells to flow into a small bioreactor of the cell expansion system.

In at least one example embodiment, a rate at which the T-cells flow into the small bioreactor may be greater than or equal to about 0.01 mL/min to less than or equal to about 100 mL/min.

In at least one example embodiment, a rate at which the T-cells flow into the small bioreactor may be greater than or equal to about 0.007 u L/min/fiber to less than or equal to about 0.0281 μL/min/fiber.

In at least one example embodiment, the method may further include maintaining the T-cells within the small bioreactor using counterflow containment.

In at least one example embodiment, the counterflow containment may include a first flow rate in a first direction and a second flow rate in an opposing second direction, where the first flow rate is about two times the second flow rate.

In at least one example embodiment, the first flow rate may be about 0.02 mL/min and the second flow rate is about 0.01 mL/min.

In at least one example embodiment, the method may further include separating T-cells from a source.

In at least one example embodiment, the separating of the T-cells from the source may include contacting magnetically conjugate antibodies to the source, the magnetically conjugate antibodies selected to associate with non-target cells or materials or components of the source; contacting non-conjugate antibodies to the source, the non-conjugate antibodies selected to associated with the T-cells; and causing the source including the magnetically conjugate antibodies and the non-conjugate antibodies to move through a magnetic column that is selected to retain the magnetically conjugate antibodies while allowing the non-conjugate antibodies to pass therethrough.

In at least one example embodiment, the method may further include harvesting produced T-cells from the bioreactor.

In at least one example embodiment, a total viable cell flown into the small bioreactor may be greater than or equal to about 20 milliliters to less than or equal to about 60 milliliters, a total variable cell of the harvested T-cells may be greater than or equal to about 100 milliliters to less than or equal to about 250 milliliters, and a percent cell viability at harvest may be greater than or equal to about 70% to less than or equal to about 100%.

In various aspects, the present disclosure provides a method for producing T-cells using a cell expansion system having a tubing aet.

In at least one example embodiment, the method may include expanding T-cells using a small bioreactor of the cell expansion system, the small bioreactor being in fluid communication with the tubing set and a volume ratio of the tubing set to the bioreactor is about 2.96.

In at least one example embodiment, the small bioreactor may have a surface area of about 2,000 cm² and an intracapillary volume of about 58 milliliters.

In at least one example embodiment, the method may further include causing T-cells to flow into a small bioreactor of the cell expansion system at greater than or equal to about 0.007 μL/min/fiber to less than or equal to about 0.0281 μL/min/fiber.

In at least one example embodiment, the method may further include maintaining the T-cells within the small bioreactor using counterflow containment, the counterflow containment including a first flow rate in a first direction and a second flow rate in an opposing second direction, where the first flow rate is about two times the second flow rate.

In at least one example embodiment, the method may further include separating T-cells from a source, where the separating of the T-cells from the source includes contacting magnetically conjugate antibodies to the source, the magnetically conjugate antibodies selected to associate with non-target cells or materials or components of the source; contacting non-conjugate antibodies to the source, the non-conjugate antibodies selected to associated with the T-cells; and causing the source including the magnetically conjugate antibodies and the non-conjugate antibodies to move through a magnetic column that is selected to retain the magnetically conjugate antibodies while allowing the non-conjugate antibodies to pass therethrough.

In at least one example embodiment, the method may further include harvesting produced T-cells from the bioreactor, where a total viable cell flown into the small bioreactor is greater than or equal to about 20 milliliters to less than or equal to about 60 milliliters, a total variable cell of the harvested T-cells is greater than or equal to about 100 milliliters to less than or equal to about 250 milliliters, and a percent cell viability at harvest is greater than or equal to about 70% to less than or equal to about 100%.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is an illustration of an example cell expansion system having a bioreactor in accordance with at least one example embodiment of the present disclosure;

FIG. 2 is an illustration of an example bioreactor that shows circulation paths through the bioreactor, and which may be incorporated into a cell expansion system, like the cell expansion system illustrated in FIG. 1, in accordance with at least one example embodiment of the present disclosure;

FIG. 3 is an illustration of an example rocking device configured to be used with a cell expansion system, like the cell expansion system illustrated in FIG. 1, to move a bioreactor, like the bioreactor of FIG. 2, in accordance with at least one example embodiment of the present disclosure;

FIG. 4 is a schematic illustrating example flow paths of an example cell expansion system, like the cell expansion system illustrated in FIG. 1, in accordance with at least one example embodiment of the present disclosure;

FIG. 5 is a flowchart illustrating an example method for producing T-cells using a cell expansion system, like the cell expansion system illustrated in FIG. 1, having a small bioreactor, in accordance with at least one example embodiment of the present disclosure;

FIG. 6 is a schematic of example counterflow containment of T-cells in a bioreactor, like the bioreactor of FIG. 2, in accordance with at least one example embodiment of the present disclosure;

FIG. 7 is a flowchart illustrating an example method for separating T-cells from a source in accordance with at least one example embodiment of the present disclosure;

FIG. 8 is a flowchart illustrating an example method for expanding T-cells using a cell expansion system, like the cell expansion system illustrated in FIG. 1, having a small bioreactor, in accordance with at least one example embodiment of the present disclosure; and

FIG. 9 is a graphical demonstration illustrating a percent dissolved gas (y-axis) over a period of time (y-axis) for a small bioreactor as compared to a standard bioreactor in accordance with at least one example embodiment of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Various components are referred to herein as “operably associated.” As used herein, “operably associated” refers to components that are linked together in operable fashion and encompasses embodiments in which components are linked directly, as well as embodiments in which additional components are placed between the linked components. “Operably associated” components can be “fluidly associated.” “Fluidly associated” refers to components that are linked together such that fluid can be transported between them. “Fluidly associated” encompasses embodiments in which additional components are disposed between the two fluidly associated components, as well as components that are directly connected. Fluidly associated components can include components that do not contact fluid but contact other components to manipulate the system (e.g., a peristaltic pump that pumps fluids through flexible tubing by compressing the exterior of the tube).

Example embodiments will now be described more fully with reference to the accompanying drawings.

Cell expansions systems are cell culturing systems used to expand and differentiate cells, including both adherent and non-adherent cell types. The present disclosure relates to cell expansion systems and processes, including, for example, cell expansion systems and process like those described in U.S. application Ser. No. 15/943,536, titled EXPANDING CELLS IN A BIOREACTOR, filed Apr. 2, 2018, and published Oct. 2, 2018 and/or U.S. Pat. No. 10,577,585 titled CELL EXPANSION and issued on Mar. 3, 2020, the entire disclosures of which are hereby incorporated by reference.

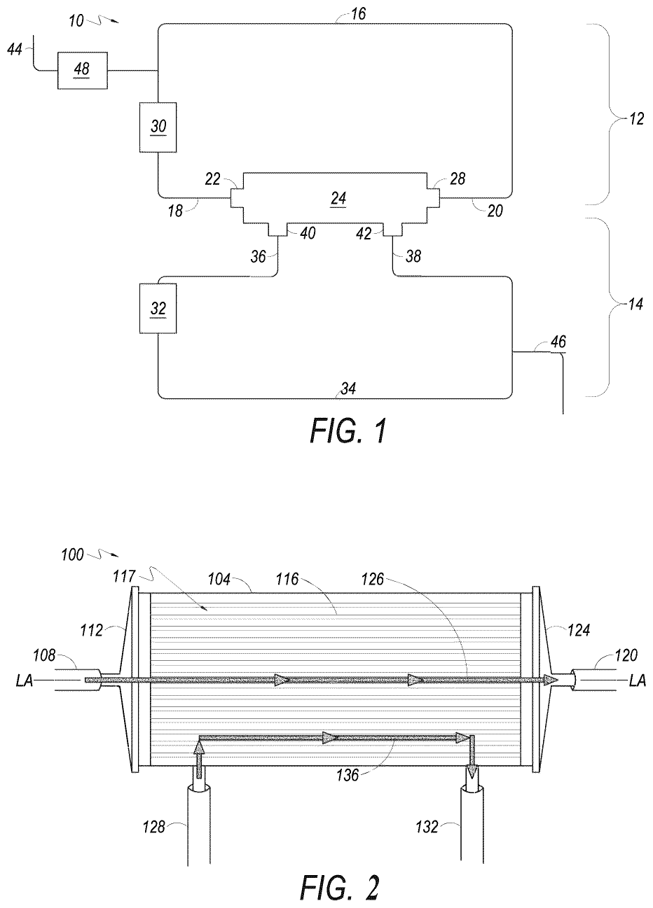

FIG. 1 is an illustration of an example cell expansion system 10. The cell expansion system 10 includes a first fluid circulation path 12 and a second fluid circulation path 14. The first fluid circulation path 12 includes, for example, a first fluid flow path 16 having opposing ends 18 and 20. The first fluid flow path 16 may be in fluid communication with a cell growth chamber 24. For example, the first opposing end 18 of the first fluid flow path 16 may be in fluid communication with a first inlet 22 of the cell growth chamber 24, and the second opposing end 20 may be in fluid communication with first outlet 28 of the cell growth chamber 24. The cell growth chamber 24 may include, or be configured to receive, a bioreactor (which may also be referred to as a hollow fiber membrane (HFM)) 117 (see FIG. 2). The bioreactor 117 may be a standard bioreactor or a small bioreactor. The standard and small bioreactors may be similarly configured and received by the same cell expansion system 10. The standard and small bioreactors, however, have different general dimensions. A standard bioreactor may be generally selected to accommodate larger cell seeds (e.g., greater than 50 M cells) and/or to produce larger cell harvests (e.g., greater than 2 B cells), while a small bioreactor may be generally selected to accommodate smaller cell seeds (e.g., less than or equal to 50 M Cells) and/or to product smaller cell harvests (e.g., less than 3 B cells) and/or to maintain lower costs. In each instance, fluid in the first circulation path 12 may flow through an interior of a plurality of hollow fibers 116 of the bioreactor 117. In at least one example embodiment, a first fluid flow control device 30 may be operably coupled to the first fluid flow path 16 and may control the flow of fluid in first fluid circulation path 12.

A small bioreactor may have an overall length that is less than an overall length of the standard bioreactor. For example, in at least one example embodiment, the small bioreactor may be half as long as the standard bioreactor. The small bioreactor may have a surface area that is less than a surface area of the standard bioreactor. For example, in at least one example embodiment, the small bioreactor may have a surface area that is about one tenth of the surface area of the standard bioreactor. In at least one example embodiment, a standard bioreactor may have a surface area of greater than or equal to about 17,000 cm² to less than or equal to about 21,000 cm², while a small bioreactor has a surface area of about 2,000 cm². The small bioreactor may have an intracapillary volume that is less than an intracapillary volume of the standard bioreactor. For example, in at least one example embodiment, the smaller bioreactor may have an intracapillary volume that is about one tenth of an intracapillary volume of the standard bioreactor. In at least one example embodiment, a standard bioreactor may have an intracapillary volume of greater than or equal to about 158 milliliters to less than or equal to about 190 milliliters, while the small bioreactor has an intracapillary volume of about 58 milliliters. In at least one example embodiment, the small bioreactor may have a ratio of tubing to bioreactor volume of about 2.96.

The second fluid circulation path 14 includes, for example, a second fluid flow path 34 and a second fluid flow control device 32. Like the first fluid flow path 16, the second fluid flow path 34 may have opposing ends 36 and 38. The opposing ends 36 and 38 of second fluid flow path 34 may in fluid communication with an inlet port 40 and an outlet port 42 of the cell growth chamber 24. For example, a first opposing end 36 of the second fluid flow path 34 may be in fluid communication with the inlet port 40 of the cell growth chamber 24, and the second opposing end 38 of the second fluid flow path 34 may be in fluid communication with the outlet port 42. Fluid in the second circulation path 14 may be in contact with an outside of the bioreactor 117 disposed in the cell growth chamber 24. In at least one example embodiment, a second fluid flow control device 32 may be operably coupled to the second fluid flow path 34 and may control the flow of fluid in the second fluid circulation path 14.

The first and second fluid circulation paths 12, 14 may be maintained in the cell growth chamber 24 by way of the bioreactor 117, where fluid in first fluid circulation path 12 flows through an intracapillary (IC) space of the bioreactor 117 and fluid in the second circulation path 14 flows through the extracapillary (EC) space of the cell growth chamber 24. The first circulation path 12 may be referred to as the “intracapillary loop” or “IC loop”. The second fluid circulation path 14 may be referred to as the “extracapillary loop” or “EC loop”. Fluid in first fluid circulation path 12 may flow in either a co-current or counter-current direction with respect to a fluid flow in second fluid circulation path 14.

In at least one example embodiment, a fluid inlet path 44 may be fluidly associated with the first fluid circulation path 12, and a fluid outlet path 46 may be fluidly associated with the second fluid circulation path 14. The fluid inlet path 44 may permit fluid into first fluid circulation path 12, while the fluid outlet path 46 may permit fluid to exit the cell expansion system 10. In at least one example embodiment, as illustrated, a third fluid flow control device 48 may be operably associated with the fluid inlet path 44. Although not illustrated, it should be recognized that, in various other example embodiments, a fourth fluid flow control device may alternatively or additionally be associated operably associated with the first outlet path 46. In at least one example embodiment, the fluid flow control devices (including the first fluid flow control device 30 and/or the second fluid flow control device 32 and/or the third fluid flow control device 48 and/or the fourth fluid flow control device) may include a pump, valve, clamp, or any combination thereof. For example, multiple pumps, valves, and clamps can be arranged in any combination. In at least one example embodiment, the fluid flow control device may be, or include, a peristaltic pump. Fluid circulation paths (including the first fluid circulation path 12 and/or the second fluid circulation path 14) and/or inlet ports (including the fluid inlet port 44) and/or the outlet port (including the fluid outlet port 46) may include any known tubing material, and any kind of fluid-including, for example, buffers, protein containing fluid, and cell-containing fluid—can flow through the various circulation paths (including the first fluid circulation path 12 and/or the second fluid circulation path 14) and/or the inlet paths (including the fluid inlet port 44), and outlet paths (including the fluid outlet port 46). It should be recognized that the terms “fluid,” “media,” and “fluid media” are used interchangeably.

An example cell growth chamber 100 is illustrated in FIG. 2. The cell growth chamber 100 may be used as the cell growth chamber 24 of the cell expansion system 10 illustrated in FIG. 1. The cell growth chamber 100 may have a longitudinal axis (represented by the line LA-LA) and may include a cell growth chamber housing 104. The cell growth chamber housing 104 may have four openings or ports, including, for example, an intracapillary inlet port 108, an intracapillary outlet port 120, an extracapillary inlet port 128, and an extracapillary outlet port 132. A first fluid (which can also be referred to as an intracapillary fluid or media) in a first circulation path (like the first fluid circulation path 12) can enter the cell growth chamber 100 through the intracapillary inlet port 108 at a first fluid manifold end 112 of the cell growth chamber 100 and into and through the intracapillary spaces of a plurality of hollow fibers 116 and out of cell growth chamber 100 through intracapillary outlet port 120, which is located at a second fluid manifold end 124 of the cell growth chamber 100. The fluid path between the intracapillary inlet port 108 and the intracapillary outlet port 120 may define an intracapillary portion 126 of the cell growth chamber 100. A second fluid (which can also be referred to as an extracapillary media or fluid) in a second circulation path (like the second fluid circulation path 14) can enter the cell growth chamber 100 through the extracapillary inlet port 128. This second fluid contacts the extracapillary space or outside of the bioreactor 117 and exits the cell growth chamber 100 via the extracapillary outlet port 132. The fluid path between the extracapillary inlet port 128 and the extracapillary outlet port 132 may define an extracapillary portion 136 of the cell growth chamber 100.

As the second fluid comes into contact with the outside of the hollow fibers 116 small molecules (e.g., ions, water, oxygen, lactate, etc.) may diffuse through the hollow fibers 116 from the interior or intracapillary space of the hollow fibers 116 to the exterior or extracapillary space, or alternatively, or additionally, from the extracapillary space to the intracapillary space. Large molecular weight molecules (e.g., growth factors and/or proteins) are often too large to pass through the membrane walls of the hollow fibers 116 and remain in the intracapillary space (or alternatively, or additionally, in the extracapillary space) of the hollow fibers 116. The mediums defining the first and second fluids may be replaced as needed and may alternatively, or additionally, be circulated through an oxygenator and/or gas transfer module to exchange gasses, as needed. As discussed below, cells for expansion (e.g., T-cells) may be contained within the first fluid circulation path 12 and/or the second fluid circulation path 14 and may enter the cell growth chamber 100 on one or both of the intracapillary space or the extracapillary space.

In at least one example embodiment, cells (e.g., T-cells) may be seeded, for example, for expansion and/or differentiation and/or harvesting in the intracapillary space of the hollow fibers 116, while a cell culture medium may be pumped through the extracapillary space of the hollow fibers 116 to deliver nutrients to the cells via hollow fiber membrane perfusion during expansion. However, in at least one other example embodiment, cells (e.g., T-cells) for expansion and/or differentiation and/or harvesting may be seeded in the extracapillary space, while the cell culture medium may be pumped through the intracapillary space to deliver nutrients to the cells via hollow fiber membrane perfusion during expansion. In at least one other example embodiment, cells (e.g., T-cells) for expansion and/or differentiation and/or harvesting may be seeded in the intracapillary space, while the cell culture medium may be pumped through both the extracapillary space and the intracapillary space. Movement of the cell culture medium through the intracapillary space and/or the extracapillary space can help to remove excess cells, for example, those not adhered to surfaces of the hollow-fiber membrane. In at least one example embodiment, the material used to form the bioreactor 117 may be any biocompatible polymeric material that is capable of being made into the hollow fibers 121. For example, synthetic polysulfone-based materials (e.g., polyethersulfones (PES)) are often used to form the hollow fibers.

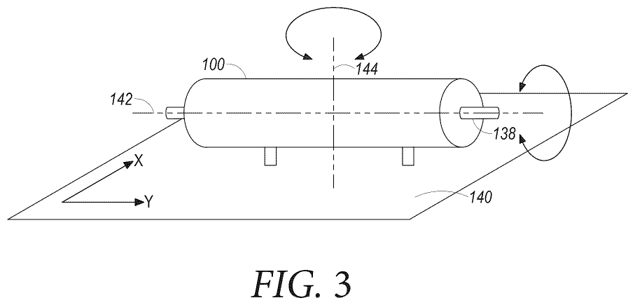

In at least one example embodiment, the cell expansion system 10 may also include a device that is configured to move or “rock” the cell growth chamber 100 relative to other components of the cell expansion system 10. The device may be a rotational and/or lateral rocking device. For example, as illustrated in FIG. 3, the cell growth chamber 100 may be rotationally connected to one or more rotational rocking components 138 and to a lateral rocking component 140. A first rotational rocking component 138 may be rotationally associated with the cell growth chamber 100. For example, the first rotational rocking component 138 may be configured to rotate the cell growth chamber 100 around a first or central rotational axis 142. In at least one example embodiment, the cell growth chamber 100 may be rotated in alternating fashion, including, for example, in a first clockwise direction and then in a second counterclockwise direction around the central axis 142.

Although not illustrated, it should be recognized that in at least one example embodiment, a second rotational rocking component may be configured to move the cell growth chamber 100 about a second rotational axis 144 that passes through a center point of the cell growth chamber 100 normal to the central axis 142. In at least one example embodiment, the cell growth chamber 100 may be rotated in alternating fashion, including, for example, in a first clockwise direction and then in a second counterclockwise direction around the second axis 144. In at least one example embodiment, the cell growth chamber 100 may also be rotated around the second axis 144 and positioned in a horizontal or vertical orientation relative to gravity. The lateral rocking component 140 may be laterally associated with the cell growth chamber 100. For example, a plane of the lateral rocking component 140 may move laterally in the x-direction and y-direction.

The rotational and/or lateral movement of the cell growth chamber 100 may reduce the settling of cells and the likelihood of cells becoming trapped within a portion of the bioreactor 117 disposed in the cell growth chamber 100. In at least one example embodiment, the rate of cells settling in the cell growth chamber 100 may be proportional to the density difference between the cells and the suspension media, according to Stoke's Law. In at least one example embodiment, a 180-degree rotation (fast) with a pause (having, for example, a total combined time of 30 seconds) repeated as described above may help to keep non-adherent cells (for example, T-cells) suspended. A minimum rotation of about 180-degrees may be preferred, however various degrees of rotation, including up to or greater than 360-degrees, may be used. Different rocking components may be used separately or may be combined in any combination. For example, a rocking component that rotates cell growth chamber 100 around central axis 142 may be combined with the rocking component that rotates cell growth chamber 100 around axis 144. Likewise, clockwise and counterclockwise rotation around different axes may be performed independently in any combination.

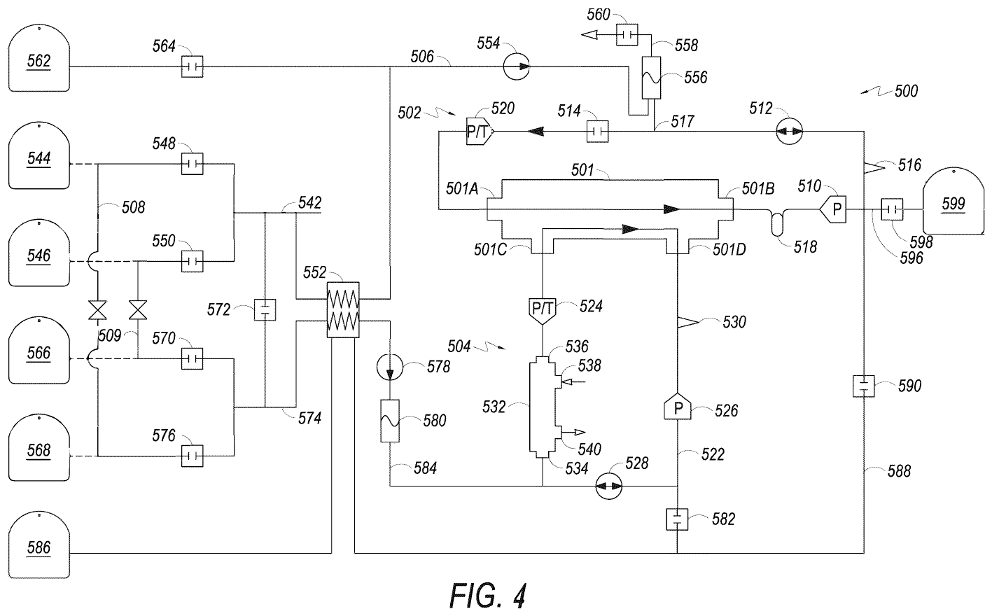

FIG. 4 is a schematic of an example cell expansion system 500, which may be like the cell expansion system 100 illustrated in FIG. 1, that illustrates example flow paths. In at least one example embodiment, the cells may be positioned in the intracapillary space, while a cell culture medium may be pumped through the extracapillary space to deliver nutrients to the cells via hollow fiber membrane perfusion during expansion. It should be recognized, however, in at least one other example embodiment, cells can be positioned in the extracapillary space, while the cell culture medium may be pumped through the intracapillary space to deliver nutrients to the cells via hollow fiber membrane perfusion during expansion. In at least one other example embodiment, cells may be positioned in the intracapillary space, while the cell culture medium may be pumped through both the extracapillary space and the intracapillary space.

As illustrated, the cell expansion system 500 may include a first fluid circulation path 502 (also referred to as the “intracapillary loop” or “IC loop”) and a second fluid circulation path 504 (also referred to as the “extracapillary loop” or “EC loop”). The first fluid flow path 506 may be fluidly associated with a cell growth chamber 501 to form first fluid circulation path 502. The cell growth chamber 501 may be used as the cell growth chamber 24 illustrated in FIG. 1 and/or the cell growth chamber 100 illustrated in FIG. 2. A first fluid may flow into cell growth chamber 501 through an intracapillary inlet port 501A. The first fluid may exit the cell growth chamber via an intracapillary outlet port 501B. In at least one example embodiment, the first fluid circulation path 502 may include a pressure gauge 510 configured to measure a pressure of the first fluid leaving the cell growth chamber 501. In at least one example embodiment, the first fluid circulation path 502 may include an intracapillary circulation pump 512 configured to control a first fluid flow rate. For example, the intracapillary circulation pump 512 may be configured to pump the first fluid in a first direction or a second direction that is opposite to the first direction. In the later instance, the intracapillary outlet port 501B may be used as an inlet, and the intracapillary inlet port 501A as an outlet. In at least one example embodiment, the first fluid circulation path 502 may include a sample port 516 and/or sample coil 518 configured for first fluid sample extraction. In at least one example embodiment, the first fluid circulation path 502 may include a pressure/temperature gauge 520 configured to detect the pressure and/or temperature of the first fluid during operation. In at least one example embodiment, the first fluid may enter the intracapillary loop 502 via valve 514. In at least one example embodiment, a portion of the cells may be flushed from the intracapillary loop 502 into a harvest bag 599, for example, via valve 598. It should be recognized that, in at least one other example embodiment, the first fluid circulation path 502 may include additional or fewer valves, pressure gauges, pressure sensors, temperature sensors, ports, and/or other devices disposed to isolate and/or measure characteristics of the first fluid along portions of the intracapillary loop 502.

A second fluid may flow into cell growth chamber 501 through an extracapillary inlet port 501C. The second fluid may leave the cell growth chamber 501 via an extracapillary outlet port 501D. In at least one example embodiment, the second fluid in the extracapillary loop 504 may contact an exterior facing surface of hollow fibers disposed in the cell growth chamber 501 thereby allowing diffusion of small molecules into and out of the hollow fibers. In at least one example embodiment, the extracapillary loop 504 may include a pressure/temperature gauge 524 configured to measure a pressure and/or temperature of the second fluid before the second fluid enters the cell growth chamber 501. In at least one example embodiment, the extracapillary loop 504 may include a pressure gauge 526 that is configured to measure a pressure of the second fluid, for example, as it leaves the cell growth chamber 501. In at least one example embodiment, the extracapillary loop 504 may include a sample port 530 configured for second fluid sample extraction.

In at least one example embodiment, the extracapillary loop 504 may include an extracapillary circulation pump 528 and an oxygenator or gas transfer module 532. For example, after leaving the cell growth chamber 501, the second fluid may pass through the extracapillary circulation pump 528 and to and through the oxygenator or gas transfer module 532. In at least one example embodiment, the extracapillary circulation pump 528 may be configured to control a second fluid flow rate. For example, like the intracapillary circulation pump 512, the extracapillary circulation pump 528 may be configured to pump the second fluid in a first direction or a second direction that is opposite to the first direction. In the later instance, the extracapillary outlet port 501D may be used as inlet, and the extracapillary inlet port 501C as an outlet.

In at least one example embodiment, the second fluid flow path 522 may be fluidly associated with the oxygenator or gas transfer module 532 via an oxygenator inlet port 534 and an oxygenator outlet port 536. For example, the second fluid may flow into the oxygenator or gas transfer module 532 via the oxygenator inlet port 534 and may leave or exit the oxygenator or gas transfer module 532 via the oxygenator outlet port 536. In at least one example embodiment, the oxygenator or gas transfer module 532 may be configured to add oxygen to and/or remove bubbles from the second fluid. For example, air and/or gas may flow into the oxygenator or gas transfer module 532 via a first filter 538 and may leave or exit (i.e., flow out of) the oxygenator or gas transfer device 532 through a second filter 540. The first and second filters 538, 540 may be configured to reduce or prevent contaminants from entering the oxygenator or gas transfer module 532. The second fluid in the second fluid circulation path 504 may be in equilibrium with gas entering the oxygenator or gas transfer module 532. In at least one example embodiment, air and/or gas may be purged from the cell expansion system 500, for example, during a priming sequence, air and/or gas may be vented to the atmosphere via the oxygenator or gas transfer module 532. It should be recognized that, in at least one other example embodiment, a second fluid circulation path 504 may include additional or fewer valves, pressure gauges, pressure sensors, temperature sensors, ports, and/or other devices disposed to isolate and/or measure characteristics of the second fluid along portions of the extracapillary loop 504.

In at least one example embodiment, an air removal chamber (ARC) 556 may be fluidly associated with the first circulation path 502. The air removal chamber 556 may include one or more ultrasonic sensors. For example, the air removal chamber 556 may include upper sensor and/or lower sensor which are configured to detect air and/or a lack of fluid and/or gas-fluid interface at certain measuring positions within the air removal chamber 556. The upper sensor may be disposed near a first end (e.g., top) of the air removal chamber 556. The lower sensor may be disposed near a second end (e.g., bottom) of the air removal chamber 556. Although ultrasonic sensors are discussed, it should be appreciated that the air removal chamber 556 may include, additionally, or alternatively, one or more other sensors, including, for example, optical sensors. Air and/or gas purged from the cell expansion system 500 during portions of a priming sequence and/or other protocols may vent to the atmosphere out air valve 560 via line 558 that may be fluidly associated with air removal chamber 556.

In at least one example embodiment, the first fluid may include cells (for example, from a first fluid container (which can also be referred to as a first media bag or a first bag) 562 and also fluid media (e.g., intracapillary media or fluid) from a second fluid container (which can also be referred to as a second media bag or a second bag) 546. Materials (i.e., cells and/or intracapillary media) form the first and second fluid containers 562, 546 may enter the first fluid circulation path 502 via a first fluid flow path 506. The first fluid container 562 may be fluidly associated with the first fluid flow path 506 and the first fluid circulation path 502 via valve 564. In at least one example embodiment, the second fluid container 546 and a third fluid container (which can also be referred to as a third media bag or third bag) 544 may be fluidly associated with the first fluid inlet path 542, for example, via valves 548 and 550, respectively, or with a second fluid inlet path 574, for example, via valves 570 and 576, respectively. In at least one example embodiment, the materials from the second fluid container 546 and/or the third fluid container 544 may be in fluid communication with a first sterile sealable input priming path 508 and/or a second sterile sealable input priming path 509.

In at least one example embodiment, a fourth fluid container (which can also be referred to as a fourth media bag or a fourth bag) 568 may include an extracapillary media, and a fifth fluid container (which can also be referred to a fifth media bag or a fifth bag) 566 may include a wash solution. Materials (i.e., extracapillary media and/or wash solution) from the fourth and fifth fluid containers 568, 566 may enter the first fluid circulation path 502 and/or the second fluid circulation path 504. For example, in at least one example embodiment, the fifth fluid container 566 may be fluidly associated with valve 570, where valve 570 is fluidly associated with first fluid circulation path 502, for example, via a distribution valve 572 and a first fluid inlet path 542. In at least one example embodiment, the fifth fluid container 566 may be fluidly associated with the second fluid circulation path 504 via the second fluid inlet path 574 and an extracapillary inlet path 584, for example, by opening valve 570 and closing distribution valve 572. The fourth fluid container 568 may be fluidly associated with valve 576, where valve 576 is fluidly associated with first fluid circulation path 502, for example, via the first fluid inlet path 542 and the distribution valve 572. In at least one example embodiment, the fourth fluid container 568 may be fluidly associated with the second fluid inlet path 574 by opening valve 576 and closing the distribution valve 572. In at least one example embodiment, the first fluid inlet path 542 and/or the second fluid inlet path 574 may be fluidly associated with an optional heat exchanger 552.

In at least one example embodiment, fluid may be advanced to the intracapillary loop 502 from the first fluid inlet path 542 and/or the second fluid inlet path 574 via an intracapillary inlet pump 554, and fluid may be advanced to the extracapillary loop 504 via an extracapillary inlet pump 578. In at least one example embodiment, an air detector 580 may also be associated with the extracapillary inlet path 584. The air detector 580 may include, for example, an ultrasonic sensor. In at least one example embodiment, the first and second fluid circulation paths 502, 504 may be fluidly associated with a waste line 588. For example, when valve 590 is in an open state or position, the intracapillary media may flow through the waste line 588 to a waste bag (also referred to as an outlet bag) 586. When valve 582 is opened, extracapillary media may flow through the waste line 588 to the waste bag 586. In at least one example embodiment, cells may be harvested, for example, via a cell harvest path 596. For example, cells from the cell growth chamber 501 may be harvested by pumping the intracapillary media containing the cells through the cell harvest path 596 and also valve 598 to a cell harvest bag 599.

In at least one example embodiment, as illustrated, the fluid in the first fluid circulation path 502 and second fluid circulation path 504 flows through cell growth chamber 501 in the same direction (i.e., a co-current configuration). Although not illustrated, it should be recognized that, in various other example embodiments, the cell expansion system 500 may also be configured to flow in a counter-current conformation. As illustrated in FIG. 4, fluid in the first fluid circulation path 502 may enter the cell growth chamber 501 at the intracapillary inlet port 501A and may leave or exit the cell growth chamber 501 at the intracapillary outlet port 501B. In at least one example embodiment, the first fluid flow path 506 may be fluidly connected to the first fluid circulation path 502, for example, via connection 517. Connection 517 may be a point or location from which the fluid may flow in opposite directions, for example, based on the direction and flow rates of the intracapillary inlet pump 554 and fluid circulation pump 512. Connection 517 may include any type of fitting, coupling, fusion, pathway, and/or tubing that allows the first fluid flow path 506 to be fluidly associated with the first fluid circulation path 502. In at least one example embodiment, connection 517 may include a T-fitting or coupling and/or a Y-fitting or coupling.

In at least one example embodiment, one or more of the gauges (e.g., pressure gauge 510 and/or pressure/temperature gauge 520 and/or pressure/temperature gauge 524 and/or pressure gauge 526), one or more of the valves (e.g., valve 514 and/or valves 548 and/or valves 550 and/or valve 560 and/or valve 564 and/or valve 570 and/or valve 572 and/or valve 576 and/or valve 582 and/or valve 590 and/or valve 596 and/or valve 598), one or more of the ports (e.g., intracapillary inlet port 501A and/or intracapillary outlet port 501B and/or extracapillary inlet port 501C and/or extracapillary outlet port 501D and/or sample port 516 and/or sample port 530 and/or oxygenator inlet port 534 and/or an oxygenator outlet port 536), one or more of the pumps (e.g., intracapillary circulation pump 512 and/or extracapillary circulation pump 528 and/or intracapillary inlet pump 554 and/or extracapillary inlet pump 578), one or more of the filters (e.g., first filter 538 and/or second filter 540), one or more coils (e.g., sample coil 518), one or more modules (e.g., oxygenator or gas transfer module 532), and/or one or more other components of the cell expansion system 500 may be in electrical communication with a control system (not shown). The control system may include a plurality of nodes, which can include various hardware, firmware, and/or software configured to control and/or communicate with the mechanical, electromechanical, and electrical components of the cell expansion system 500, including for example, a controller and a memory.

The controller (which can also be referred to as a processor), can be of any type of microcontroller, microprocessor, Field Programmable Gate Array (FPGA), Application Specific Integrated Circuit (ASIC), etc. An example controller may be the NK10DN512VOK10 microcontroller, made and sold by N9P USA, Incorporated, which is a microcontroller unit with a 32-bit architecture. Other examples controllers may include, for example, at least one of Qualcomm® Snapdragon® 800 and 801, Qualcomm® Snapdragon® 610 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i-4770K 22 nm Haswell, Intel® Core® i5-3570K 22 nm Ivy Bridge, the AMD® FX™ family of processors, AMD® FX-4300, FX-6300, and FX-8350 32 nm Vishera, AMD® Kaveri processors, ARM® Cortex™-M processors, ARM® Cortex-A and ARM926EJ-STM processors, other industry-equivalent processors, and may perform computational functions using any known or future-developed standard, instruction set, libraries, and/or architecture. The memory can be any type of memory including random access memory (RAM), read only memory (ROM), electrically erasable programmable ROM (EEPROM), a portable compact disc read-only memory (CD-ROM), an optical storage device, a magnetic storage device, any suitable combination of the foregoing, or other type of storage or memory device that stores and provides instructions to program and control the controller.

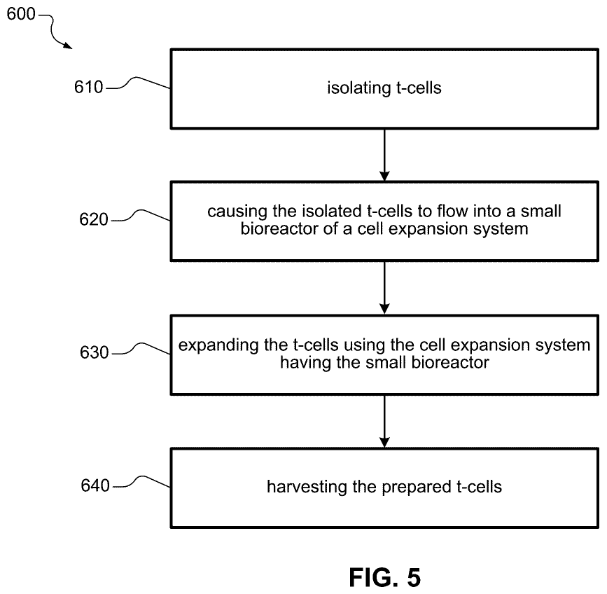

The present disclosure provides example methods for producing T-cells, for example, using a cell expansion system, like the cell expansion system 10 illustrated in FIG. 1, including a small bioreactor having, for example, a surface area of about 2,000 cm² and an intracapillary volume of about 58 milliliters. For example, FIG. 5 is a flowchart illustrating an example method 600 that includes causing 620 isolated T-cells to flow into a small bioreactor of a cell expansion system. In at least one example embodiments, the isolated T-cells may be caused to flow into an intracapillary loop of the small bioreactor. More specifically, the T-cells may be caused to flow into the intracapillary loop via an intracapillary inlet (e.g., the first inlet 22 of the cell growth chamber 24 as illustrated in FIG. 1 and/or the intracapillary inlet port 108 of the cell growth chamber 100 as illustrated in FIG. 2 and/or the intracapillary inlet port 501A of the cell growth chamber 501 as illustrated in FIG. 4) using, or adjusting, an unidirectional intracapillary inlet flow, for example, from an media bag (e.g., the second fluid container 546 of the cell expansion system 500 as illustrated in FIG. 4). In at least one example embodiment, a rate at which the T-cells flow into the small bioreactor may be greater than or equal to about 0.01 mL/min to less than or equal to about 100 mL/min, optionally greater than or equal to about 1 mL/min to less than or equal to about 100 mL/min, optionally greater than or equal to about 1 mL/min to less than or equal to about 10 mL/min, and optionally about 5 mL/min. In at least one example embodiment, a rate at which the T-cells flow into the small bioreactor may be greater than or equal to about 0.007 μL/min/fiber to less than or equal to about 0.0281 μL/min/fiber. By way of comparison, a rate at which the T-cells flow into a standard bioreactor is often greater than or equal to about 0.017 μL/min/fiber to less than or equal to about 0.0347 μL/min/fiber.

In at least one example embodiment, a total viable cell amount flown into the small bioreactor may be greater than or equal to about 20 millimeters to less than or equal to about 60 millimeters, and optionally, greater than or equal to about 30 milliliters to less than or equal to about 50 milliliters.

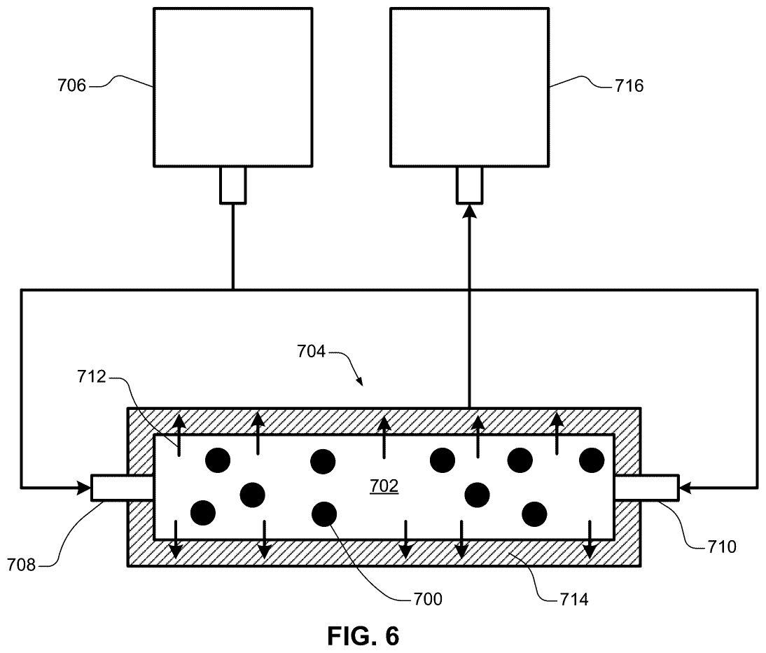

Although not illustrated, it should be appreciated that, in various example embodiments, the method 600 may include maintaining the T-cells within the small bioreactor, and more specifically, for example, in the intracapillary loop, using counterflow containment. By way of example, FIG. 6 provides a schematic of example counterflow containment of T-cells. As illustrated, T-cells (and/or other cells) 700 may be positioned (or repositioned) within an intracapillary portion or space 702 of a bioreactor 704 using counterflow containment. More specifically, as media or fluid moves (i.e., flows) from a media bag or container 706 it may be split between an intracapillary inlet pump (not shown) and an intracapillary circulation pump (not shown) such that the media or fluid moves into the intracapillary portion or space 702 of the bioreactor 704 through both an intracapillary inlet 708 and an intracapillary outlet 710. In contrast, T-cells (and/or other cells) 700 may be seeded and/or recirculated using unidirectional flow from the media bag or container 706 to the intracapillary portion or space 702 of a bioreactor 704 via the intracapillary inlet 708.

In at least one example embodiment, during a feeding stage, the intracapillary inlet flow rate is about 2 times of the intracapillary circulation pump rate. For example, the intracapillary inlet flow rate may be greater than or equal to about 0.02 mL/min and the intracapillary circulation pump rate may be greater than or equal to about 0.01 mL/min. Arrows 712 illustrate the fluid movement through pores of the bioreactor 704 from the intracapillary portion or space 702 to the extracapillary portion or space 714 during counterflow containment. A waste bag 716 may be in fluid communication with the extracapillary portion or space 714 allowing appropriate movement from the extracapillary portion 714 to the waste bag 716.

As discussed above, although the cells (e.g., T-cells) 200 are illustrated as being seeded within the intracapillary portion or space 702, it should be appreciated that, in various other example embodiments, the cells 200 may instead be seeded instead in the extracapillary portion or space 714. In such instances counterflow containment may include moving media or fluid from the media bag or container 706 to an extracapillary inlet (not shown) and also an extracapillary outlet (not shown).

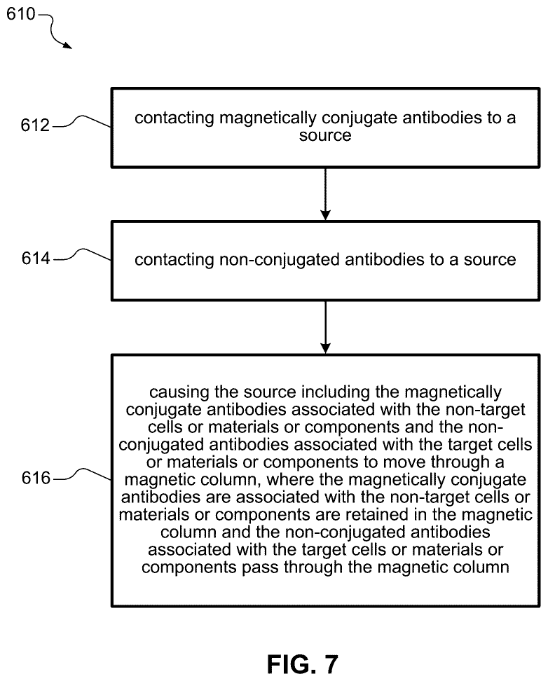

With renewed reference to FIG. 5, in at least one example embodiment, the method 600 may include isolating or separating 610 the target cells (i.e., T-cells) from a source. In at least one example embodiment, the target cells may be isolated from a source using an immunomagnetic positive selection process. In other example embodiments, the target cells may be isolated from a source using magnetically conjugated antibodies and non-conjugated antibodies. For example, FIG. 7 illustrates an example method for separating 610 T-cells from a source may include using magnetically conjugated antibodies and non-conjugated antibodies. For example, the separating of the target cells from the source may include contacting 612 the source to the magnetically conjugate antibodies and contacting 614 the source to the non-conjugated antibodies (which may also be referred to as stimulating antibodies. Although illustrated as separate steps, it should be appreciated that, in various example embodiments, the magnetically conjugate antibodies and the non-conjugated antibodies may be contacted 612, 614 to the source concurrently or subsequently. For example, in at least one example embodiment, the magnetically conjugate antibodies may be contacted 614 to the source and then the non-conjugated antibodies may be contacted 614 to the source. In other example embodiments, the non-conjugated antibodies may be contacted 614 to the source and then the magnetically conjugate antibodies may be contacted 614 to the source. In still another example embodiment, the magnetically conjugate antibodies may be contacted 614 to the source and the non-conjugated antibodies may be contacted 614 to the source at the same time. For example, in at least one example embodiment, the contacting 612 of the magnetically conjugate antibodies to the source and the contacting 614 of the non-conjugated antibodies to the source may include moving the source (including non-target cells or materials or components and the target cells) from a first bag (e.g., an inlet bag) to another bag (e.g., stimulation and selection bag), where the another bag includes the magnetically conjugate antibodies and the non-conjugated antibodies. In each instance, the magnetically conjugate antibodies are selected to bind or associate with non-target cells or materials or components and the non-conjugated antibodies are selected to bind or associate with the target cells and the contacting 612, 614 may occur for a selected time period.

After the magnetically conjugate antibodies are associated with the non-target cells and the non-conjugated antibodies are associated with the target cells, the isolating 610 may further include causing the source including the magnetically conjugate antibodies and the non-conjugated antibodies to move through a magnetic column, where the magnetic column that is configured to retain the magnetically conjugate antibodies associated with the non-target cells or materials or components while allowing the non-conjugated antibodies associated with the target cells to pass therethrough and onto the intracapillary loop (for example, such as the first circulation path 12 illustrated in FIG. 1 and/or the first fluid circulation path 502 illustrated in FIG. 4), or alternatively, or additionally, to the extracapillary loop (for example, such as the second fluid circulation path 14 illustrated in FIG. 1 and/or the second fluid circulation path 504 illustrated in FIG. 4).

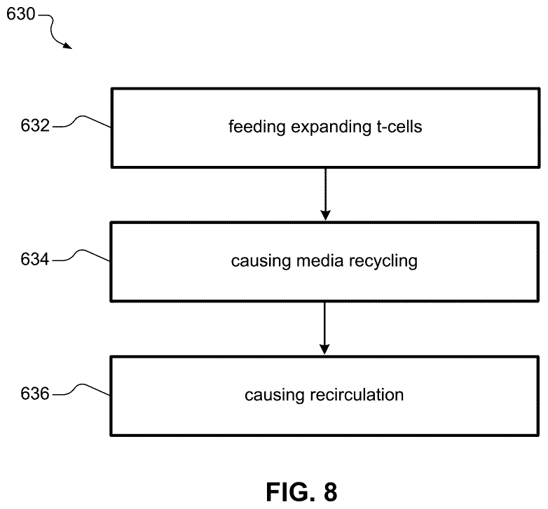

With renewed reference to FIG. 5, the method 600 may further includes expanding 630 the T-cells within the small bioreactor. In at least one example embodiment, as illustrated in FIG. 8, expanding 630 the T-cells within the small bioreactor may include feeding 632 the expanding T-cells, for example by moving a cell culture medium through the small bioreactor, and more particularly, the extracapillary space when the T-cells are loaded in the intracapillary space, such as detailed above. Expanding 630 the T-cells within the small bioreactor may further include causing 634 media recycling. In at least one example embodiment, media recycling may include establishing fluid communication between a waste bag or container (e.g., waste bag 586 of the cell expansion system 500 as illustrated in FIG. 4 and/or the waste bag 716 illustrated in FIG. 6) and to an extracapillary media bag (e.g., the fourth fluid container 568 of the cell expansion system 500 as illustrated in FIG. 4) to create an extracapillary media loop through the bioreactor. Expanding 630 the T-cells within the small bioreactor may further includes causing 636 recirculation. In at least one example embodiment, media recirculation may include the recirculation of the media through the small bioreactor, for example, prior to counterflow containment and perfusion feeding of the cells on a predetermined or selected basis (e.g., daily).

With renewed reference to FIG. 5, the method 600 may further include harvesting 640 the prepared T-cells from the small bioreactor. In at least one example embodiment, the harvesting 640 may include moving the cells within an infusion of media from the bioreactor to a harvest bag (e.g., like harvest bag 599 illustrated in FIG. 4), for example, by opening, or causing to open, one or move valves and allowing the cell suspension to travel to the harvest bag. In at least one example embodiment, a total variable cell of the harvested T-cells may be greater than or equal to about 100 milliliters to less than or equal to about 250 millimeters. In at least one example embodiment, a percent cell viability at harvest may be greater than or equal to about 70% to less than or equal to about 100%.

Embodiments of the present disclosure are further illustrated through the following non-limiting examples.

### Example 1

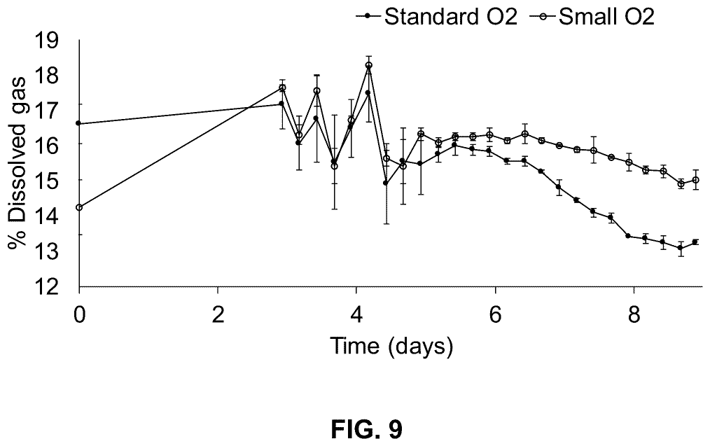

In a first instances, primary donor T-cells were loaded into a small bioreactor having, for example, a surface area of about 2,000 cm² and an intracapillary volume of about 58 milliliters, with soluble anti-CD2/anti-CD3/anti-CD28 antibody activators and expanded with Prime XV T-cell XSFM media. In a second instance, primary donor T-cells were activated using the same activators (i.e., soluble anti-CD2/anti-CD3/anti-CD28 antibody activators) but grown using a flask culture. In a third instance, primary donor T-cells were loaded into a standard bioreactor having, for example, a surface area of greater than or equal to about 17,000 cm² to less than or equal to about 21,000 cm² and an intracapillary volume of greater than or equal to about 158 milliliters to less than or equal to about 190 milliliters, with the same activators (i.e., soluble anti-CD2/anti-CD3/anti-CD28 antibody activators) and expanded with the same media (i.e., Prime XV T-cell XSFM media). In the first instance, the small bioreactor generated between about 22.2% and 33.1% of Tₛₑₘ after eight days, while in the second instance, the flask culture generated between about 1.8% and 3.7% of Tₛₑₘ after the same time period, and in the third instance, the standard bioreactor generated less than about 4% of Tₛₑₘ after the same time period. Although not yet fully understood, it is believed that the lower intracapillary inlet flow rates and/or the higher level of concentrated culture media in the small bioreactor as compared to the standard bioreactor surprisingly causes elevated dissolved oxygen levels in the instance of the small bioreactor as compared to the standard bioreactor, which surprisingly results in the higher generated Tₛₑₘ. For example, FIG. 9 illustrates the percent dissolved gas for the small bioreactor as compared to the standard bioreactor over the selected time period (i.e., eight days).

### Example 2

In a fourth instance, primary donor T-cells were loaded into a small bioreactor having, for example, a surface area of about 2,000 cm² and an intracapillary volume of about 58 milliliters, with soluble anti-CD2/anti-CD3/anti-CD28 antibody activators and expanded with X-Vivo 15 (with modifications to the number of circulations to accommodate the higher protein concentration in the media as compared to Example 1). In a fifth instance, primary donor T-cells were activated using the same activators (i.e., soluble anti-CD2/anti-CD3/anti-CD28 antibody activators) but grown using a flask culture. In a sixth instance, primary donor T-cells were loaded into a standard bioreactor having, for example, a surface area of greater than or equal to about 17,000 cm² to less than or equal to about 21,000 cm² and an intracapillary volume of greater than or equal to about 158 milliliters to less than or equal to about 190 milliliters, with the same activators i.e., soluble anti-CD2/anti-CD3/anti-CD28 antibody activators) and expanded with the same media (i.e., X-Vivo 15). In the fourth instance, the small bioreactor generated between about 44.4% and about 46.8% of Tₛₑₘ after nine days, while in the fifth instance, the flask culture generated between about 10.3% and about 14.9% of Tₛₑₘ after the same time period, and in the sixth instance, the standard bioreactor generated less than about 15% of Tₛₑₘ after the same time period.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for producing T-cells using a cell expansion system, the method comprising:
expanding T-cells using a small bioreactor of the cell expansion system, the small bioreactor having a surface area of about 2,000 cm².

2. The method of claim 1, wherein the small bioreactor has an intracapillary volume of about 58 milliliters.

3. The method of claim 1, wherein the cell expansion system further includes a tubing set that is in fluid communication with the small bioreactor and a volume ratio of the tubing set to the bioreactor is about 2.96.

4. The method of claim 3, wherein the method further includes:
placing the tubing set into fluid communication with the small bioreactor.

5. The method of claim 1, wherein the method further includes:
causing T-cells to flow into a small bioreactor of the cell expansion system.

6. The method of claim 5, wherein a rate at which the T-cells flow into the small bioreactor is greater than or equal to about 0.01 mL/min to less than or equal to about 100 mL/min.

7. The method of claim 5, wherein a rate at which the T-cells flow into the small bioreactor is greater than or equal to about 0.007 μL/min/fiber to less than or equal to about 0.0281 μL/min/fiber.

8. The method of claim 1, wherein the method further includes:
maintaining the T-cells within the small bioreactor using counterflow containment.

9. The method of claim 8, wherein
the counterflow containment includes a first flow rate in a first direction and a second flow rate in an opposing second direction, and
the first flow rate is about two times the second flow rate.

10. The method of claim 9, wherein the first flow rate is about 0.02 mL/min and the second flow rate is about 0.01 mL/min.

11. The method of claim 3, wherein the method further includes:
separating T-cells from a source.

12. The method of claim 11, wherein the separating of the T-cells from the source includes:
contacting magnetically conjugate antibodies to the source, the magnetically conjugate antibodies selected to associate with non-target cells or materials or components of the source;
contacting non-conjugate antibodies to the source, the non-conjugate antibodies selected to associated with the T-cells; and
causing the source including the magnetically conjugate antibodies and the non-conjugate antibodies to move through a magnetic column that is selected to retain the magnetically conjugate antibodies while allowing the non-conjugate antibodies to pass therethrough.

13. The method of claim 1, wherein the method further includes:
harvesting produced T-cells from the bioreactor.

14. The method of claim 13, wherein
a total viable cell flown into the small bioreactor is greater than or equal to about 20 milliliters to less than or equal to about 60 milliliters,
a total variable cell of the harvested T-cells is greater than or equal to about 100 milliliters to less than or equal to about 250 milliliters, and
a percent cell viability at harvest is greater than or equal to about 70% to less than or equal to about 100%.

15. A method for producing T-cells using a cell expansion system having a tubing set, the method comprising:
expanding T-cells using a small bioreactor of the cell expansion system, the small bioreactor being in fluid communication with the tubing set and a volume ratio of the tubing set to the bioreactor is about 2.96.

16. The method of claim 15, wherein the small bioreactor having a surface area of about 2,000 cm² and an intracapillary volume of about 58 milliliters.

17. The method of claim 15, wherein the method further includes:
causing T-cells to flow into a small bioreactor of the cell expansion system at greater than or equal to about 0.007 μL/min/fiber to less than or equal to about 0.0281 μL/min/fiber.

18. The method of claim 15, wherein the method further includes:
maintaining the T-cells within the small bioreactor using counterflow containment, the counterflow containment including a first flow rate in a first direction and a second flow rate in an opposing second direction, the first flow rate being about two times the second flow rate.

19. The method of claim 15, wherein the method further includes:
separating T-cells from a source, wherein the separating of the T-cells from the source includes:
contacting magnetically conjugate antibodies to the source, the magnetically conjugate antibodies selected to associate with non-target cells or materials or components of the source;
contacting non-conjugate antibodies to the source, the non-conjugate antibodies selected to associated with the T-cells; and
causing the source including the magnetically conjugate antibodies and the non-conjugate antibodies to move through a magnetic column that is selected to retain the magnetically conjugate antibodies while allowing the non-conjugate antibodies to pass therethrough.

20. The method of claim 15, wherein the method further includes:
harvesting produced T-cells from the bioreactor, wherein
a total viable cell flown into the small bioreactor is greater than or equal to about 20 milliliters to less than or equal to about 60 milliliters,
a total variable cell of the harvested T-cells is greater than or equal to about 100 milliliters to less than or equal to about 250 milliliters, and
a percent cell viability at harvest is greater than or equal to about 70% to less than or equal to about 100%.
